(19) United States
(12) Patent Application Publication
(10) Pub. No.: US 2008/0241208 A1
(43) Pub. Date: Oct. 2, 2008

(54) Methods, Compositions and Devices For Promoting Anglogenesis

(76) Inventors: Charles Shanley, Grosse Pointe Farms, MI (US); Scott Merz, Ann Arbor, MI (US); Melissa M. Reynolds, Ann Arbor, MI (US)

Correspondence Address: GOODWIN PROCTER LLP;PATENT ADMINISTRATOR, EXCHANGE PLACE, BOSTON, MA, 02109-2881, US

(21) Appl. No.: 11/994,004

(22) PCT Filed: Jun. 30, 2006
(86) PCT No.: PCT/US06/25855

§ 371 Date: Jun. 6, 2008

Related U.S. Application Data
(60) Provisional application No. 60/695,266, filed on Jun. 30, 2005.

(51) Int. Cl. A61K 9/00; A61F 2/02; A61K 33/00; A61K 38/20; A61P 9/10

(52) U.S. Cl. 424/423; 623/1.46; 424/718; 424/85.2

(57) ABSTRACT

This disclosure is, at least in part, directed to compositions, devices, and methods of promote angiogenesis.

### RELATED APPLICATIONS

This application claims priority to U.S. Ser. No. 60/695,266 filed Jun. 30, 2005 and hereby incorporated by reference in its entirety.

### INTRODUCTION

Many individuals suffer from circulatory diseases caused by a progressive atherosclerosis that also affects the heart and other major organs. Arterial occlusive diseases and ischemic heart diseases result in 500,000-600,000 deaths in the United States annually.

Vascular stents have now been used clinically for more than a decade to treat peripheral arterial occlusive disease percutaneously. Although the use of stents relieves the initial occlusion of the vessel, the implantation procedure can also damage the fragile endothelium, leading to proliferation of underlying smooth muscle cells and ultimately vascular stenosis caused by neointimal hyperplasia. For example, in percutaneous transluminal coronary angioplasty, the endothelium undergoes virtually complete desquamation at the treatment site due to the luminally-positioned catheter-based instrumentation. Placement of metallic stents, which are used in roughly 80-90% of procedures currently, has helped reduce, but not eliminate, restenosis caused by the damage to the interior wall of vessels during angioplasty; whereas chronic constrictive vessel remodeling occurs after balloon angioplasty alone, placement of a metal stent scaffold eliminates this chronic recoil of the vessel.

In addition, the stent material itself is foreign and provides sites for protein adsorption which can lead to platelet activation and thrombus formation, exacerbating the risk of thrombosis incited by endothelial removal. This risk is typically mitigated by administering platelet inhibitors systemically during the first month following the procedure. As a result, systemic anticoagulation regimens (e.g., use of heparin for short term applications; low molecular weight heparinoids, Plavix, and other anti-platelet agents for longer term) are almost always required clinically to reduce the risk of thrombus formation. The long-term use of exogenous anticoagulants, however, can also have adverse effects, especially a greatly increased possibility of hemorrhage. In addition, even when anticoagulant levels can be managed effectively, thrombocytopenia (platelet consumption) and thrombosis can still occur. Further, there is always risk of bleeding when patients are on given anticoagulants.

Numerous approaches aimed at overcoming these problems are currently being investigated worldwide. For example, nitric oxide agents have been found that mitigate restenosis. No such agents have been found to date that promote angiogenesis.

Thus, although nitric oxide agents have been shown to mitigate restenosis, nitric oxide has not been shown to be of therapeutic benefit in promoting, or enhancing endothelial repair at sites of vascular injury. Further, a need remains for devices that reduce the potential harmful effects and increase the beneficial effects of such a device in a patient. Consequently, there is a need for compounds and/or compositions and devices that promote and/or induce such angiogenesis.

### SUMMARY OF THE INVENTION

It is one object of the invention to provide compositions, devices and methods for creating, promoting and/or inducing blood vessel formation and/or angiogenesis and/or endothelial repair.

In one aspect of this disclosure, nitric oxide agents and compositions and/or devices are provided that promote angiogenesis to a patient in need thereof. Further, methods are provided herein for promoting and inducing angiogenesis by administering a nitric oxide agent to a patient in need thereof.

Methods are provided herein for promoting blood vessel formation in hypoxic or ischemic tissue in a patient in need thereof, comprising contacting said tissue with a biocompatible composition comprising a nitric oxide releasing agent or nitric oxide generating agent thereby generating an effective amount of nitric oxide to said tissue. In some embodiments, the composition promotes blood vessel formation by promoting angiogenesis. The disclosed compositions may promote blood vessel formation by promoting formation or maturation of collateral blood vessels. In some embodiments, the tissue is cardiac tissue, neural tissue, muscle, skin, bone, or visceral organ tissue.

Methods are also provided for promoting angiogenesis in a subject in need thereof, comprising implanting a biocompatible composition comprising a nitric oxide releasing agent or nitric oxide generating agent into said subject at a tissue locus thereby generating an effective amount of nitric oxide to said tissue. The nitric oxide releasing agent may be a diazeniumdiolate. A nitric oxide generating agent may be a metal-ligand complex capable of reducing nitrosothiol species to nitric oxide, such as a metal-cyclen complex or metal-cyclam complex, a copper-cyclen complex, copper-cyclam complex, or a N₄ donor type macrocycle.

In some embodiments nitric oxide is generated or released by the disclosed compositions and diffuse at least 10 microns, through tissue, away from a surface of the composition. The nitric oxide generated or released by a disclosed composition may diffuse at least 15, 20, 25, 35, 50, 75, or 100 microns, through tissue, away from a surface of the composition. Compositions may further comprise a polymer having said nitric oxide releasing agent or nitric oxide generating agent dispersed therein or thereon and/or a disclosed composition may further comprise a pro-angiogenic factor, such as vascular endothelial growth factor (VEGF), fibroblast growth factor (FGF), hepatocyte growth factor (HGF), platelet-derived growth factor (PDGF), interleukin 6 (IL-6), monocyte chemotactic protein 1 (MCP-1), granulocyte-macrophage colony stimulating factor (GM-CSF), or transforming growth factor β (TGFβ).

Methods disclosed herein may include a composition that is a coating or film disposed on a surface of an implantable medical device, where the device may be for example, a stent, a shunt, a pacemaker lead, an implantable defibrillator, a suture, a staple, or a perivascular wrap, or a pliable sheet or membrane, which can substantially conform to the contours of a wound site comprising said tissue.

Also disclosed herein is a method of enhancing endothelial repair at site of vascular injury, comprising administering an effective amount of nitric oxide to said site.

Methods are provided for enhancing blood perfusion in hypoxic tissue and/or ischemic tissue in a patient in need thereof, comprising contacting said tissue with a composition comprising a nitric oxide releasing agent or a nitric oxide generating agent.

In an embodiment, a method of promoting blood vessel formation in hypoxic or ischemic tissue in a patient in need thereof is provided, comprising implanting a composition comprising a nitric oxide releasing agent or nitric oxide generating agent into a subject having a locus of hypoxic or ischemic tissue. The composition may be implanted, for example, adjacent to or into the locus of hypoxic or ischemic tissue, for example, cardiac tissue, neural tissue, muscle, skin, bone, or visceral organ tissue. In some embodiments, compositions disclosed herein promote blood vessel formation by promoting angiogenesis, by for example, promoting formation or maturation of collateral blood vessels. In other embodiments, methods of promoting angiogenesis in a subject afflicted with atherosclerosis is also provided, comprising implanting a composition comprising a nitric oxide releasing agent or nitric oxide generating agent into said subject at a tissue locus experiencing or at risk of insufficient blood perfusion. In some embodiments, the subject is afflicted with coronary artery disease (CAD), congestive heart failure (CHF), is experiencing or has experienced angina pectoris, and/or is experiencing or has experienced claudication.

A method of enhancing blood perfusion in myocardial tissue of a subject in need of such enhancement is provided herein, comprising implanting a composition comprising a nitric oxide releasing agent or nitric oxide generating agent into said subject at a myocardial tissue locus experiencing insufficient blood perfusion. In some embodiments, the subject is experiencing or has experienced myocardial infarction, and the locus of implantation is ischemic myocardial tissue, is experiencing or has experienced myocardial infarction, and the locus of implantation is hibernating myocardial tissue.

In other embodiments, a method of enhancing blood perfusion in neural tissue of a subject in need of such enhancement is provided, comprising the step of implanting a composition comprising a nitric oxide releasing agent or nitric oxide generating agent into said subject at a neural tissue locus experiencing insufficient blood perfusion. In another embodiment, a method of enhancing blood perfusion in peripheral tissue of a subject in need of such enhancement, comprising the step of implanting a composition comprising a nitric oxide releasing agent or nitric oxide generating agent into said subject at a peripheral tissue locus experiencing insufficient blood perfusion. For example, a subject may be afflicted with a peripheral vascular disease, e.g., diabetes or Raynaud's disease, traumatic injury, a crush injury and said locus is distal to the site of said crush injury, or has experienced partial or complete amputation of a body part comprising said locus, or has been burned or frostbitten. In some embodiments, a nitric oxide releasing agent is a diazeniumdiolate and/or a nitric oxide generating agent is a metal-ligand complex. The nitric oxide generated or released by said composition may diffuse at least about 10 microns, 15, 20, 25, 35, 50, 75, or 100 microns, through tissue, away from a surface of said composition.

In some embodiments, a composition is a coating or film disposed on a surface of an implantable medical device, such a stent, a shunt, pacemaker lead, an implantable defibrillators, a suture or staple, a perivascular wrap, a pliable sheet or membrane. In some embodiments, an implantable composition may be the form of a pliable sheet or membrane, a malleable plug, a particle, microcapsule, or spray.

In another aspect, the subject compositions and compounds may be used in the manufacture of a medicament for any number of uses, including for example treating any disease, or other treatable condition of a patient, for example wound healing.

The embodiments and practices of the present invention, other embodiments, and their features and characteristics, will be apparent from the description, drawings and claims that follow.

### BRIEF DESCRIPTION OF THE FIGURES

FIGS. 1A and B show tissue incorporation in sham-coated (top) and NO-releasing (bottom) grafts at 21 d and 3 months post sheep implant. Diffusion of the NO resulted in reduced tissue incorporation on the abluminal surface compared to control grafts at both time points.

FIG. 2 shows the result of Factor VIII staining of a capsule surrounding an NO-releasing graft indicating incipient angiogenesis in the area surrounding the graft after 3 months. Tissue further from the graft does not show this pattern, nor does tissue surrounding the control graft. The incipient angiogenesis is oriented towards the graft.

FIG. 3 depicts an in-vitro assay matrix with a diazeniumdiolate compound pulsed with PMA, bFGF for 2 hr followed by PMA, bFGF and VEGF-C for 70 hr.

FIG. 4 depicts an in-vitro assay matrix with a diazeniumdiolate compound treated with PMA, bFGF and VEGF-C for 72 hr.

FIG. 5 depicts an in-vitro assay matrix treated with PMA, bFGF for 72 hr.

FIG. 6 depicts in vivo angiogenesis rabbit implant studies showing increased angiogenic activity using a nitric oxide generating compound.

FIG. 7 depicts in vivo angiogenesis rabbit implant studies showing increased angiogenic activity using a nitric oxide generating compound.

FIG. 8 depicts in vivo angiogenesis rabbit implant studies showing increased angiogenic activity using a nitric oxide generating compound.

### DETAILED DESCRIPTION OF THE INVENTION

### Overview

The present invention relates at least in part, to compounds, compositions, methods and devices that generate, promote, and/or induce angiogenesis in a patient. It has been discovered, at least in part, that nitric oxide agents, such as nitric oxide generating and/or donating agents, induces angiogenesis when incorporated or placed on a medical device, such as a stent. For example, methods are provided herein for promoting and inducing angiogenesis by administering a nitric oxide agent to a patient in need thereof.

### DEFINITIONS

For convenience, before further description of the present invention, certain terms employed in the specification, examples, and appended claims are collected here. These definitions should be read in light of the remainder of the disclosure and understood as by a person of skill in the art. Also, the terms “including” (and variants thereof), “such as”, “e.g.” as used herein are non-limiting and are for illustrative purposes only.

The articles “a” and “an” are used herein to refer to one or to more than one (i.e. to at least one) of the grammatical object of the article. By way of example, “an element” means one element or more than one element.

The term “angiogenesis” is an art-recognized term, and refers to the process and creation of new blood vessels formed from pre-existing blood vessels.

The term “restenosis” refers to a re-narrowing of a blood vessel, thereby restricting blood flow. This re-narrowing can be caused by, for example, a vessel's response to an injury inflicted during balloon angioplasty.

The term “hypoxic tissue” refers to tissue with an insufficient amount of oxygen. The term “ischemic tissue” refers to tissue with insufficient blood flow.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in 1M NaOH at 37° C. until complete degradation is observed. The solution is then neutralized with 1M HCl. About 200 μL of various concentrations of the degraded sample products are placed in 96-well tissue culture plates and seeded with human gastric carcinoma cells (GT3TKB) at 10⁴/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The term “drug delivery device” is an art-recognized term and refers to any medical device suitable for the application of a drug or therapeutic agent to a targeted organ or anatomic region. The term includes, without limitation, those formulations of the compositions of the present invention that deliver the therapeutic agent into the surrounding tissues of an anatomic area. The term further includes those devices that transport or accomplish the instillation of the compositions of the present invention towards the targeted organ or anatomic area, even if the device itself is not formulated to include the composition. As an example, a needle or a catheter through which the composition is inserted into an anatomic area or into a blood vessel or other structure related to the anatomic area is understood to be a drug delivery device. As a further example, a stent or a shunt or a catheter that has the composition included in its substance or coated on its surface is understood to be a drug delivery device.

The term “delivery agent” is an art-recognized term, and includes molecules that facilitate the intracellular delivery of a therapeutic agent or other material. Examples of delivery agents include: sterols (e.g., cholesterol) and lipids (e.g., a cationic lipid, virosome or liposome).

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and includes relatively non-toxic, inorganic and organic acid addition salts of compositions of the present invention, including without limitation, nitric oxide generating agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

A “patient,” “subject,” or “host” to be treated by the subject method may mean either a human or non-human animal, such as primates, mammals, and vertebrates.

The terms “incorporated” and “encapsulated” are art-recognized when used in reference to an nitric oxide generating agent (or other material) and a polymeric composition, such as a composition of the present invention. In certain embodiments, these terms include incorporating, formulating or otherwise including such agent into a composition which allows for the prevention of biofouling and/or permits analyte diffusion of such agent in the desired application. The terms may contemplate any manner by which an nitric oxide generating agent or other material is incorporated into a polymer matrix, including for example: attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), associated with the surface of the polymer (by spraying, dipping or other methods), encapsulated inside the polymeric matrix, etc. The term “co-incorporation” or “co-encapsulation” refers to the incorporation of a nitric oxide generating agent or other material and at least one other agent or other material in a subject composition.

More specifically, the physical form in which any nitric oxide generating agent or other material is encapsulated in polymers may vary with the particular embodiment. For example, a nitric oxide generating agent or other material may be first encapsulated in a microsphere and then combined with the polymer in such a way that at least a portion of the microsphere structure is maintained. Alternatively, a nitric oxide generating agent or other material may be sufficiently immiscible in the polymer of the invention that it is dispersed as small droplets, rather than being dissolved, in the polymer. Any form of encapsulation or incorporation is contemplated by the present invention, in so much as the effectiveness over time of any encapsulated nitric oxide generating agent or other material determines whether the form of encapsulation is sufficiently acceptable for any particular use.

As used herein, the term “nitric oxide” encompasses uncharged nitric oxide and charged nitric oxide species, including for example, nitrosonium ion and nitroxyl ion.

The term “metal-ligand complex” refers to a chemical species with at least one ligand capable of coordinating to at least one central metal ion.

The term “aliphatic” is an art-recognized term and includes linear, branched, and cyclic alkanes, alkenes, or allynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain allyl has about 30 or fewer carbon atoms in its backbone (e.g., C1-C30 for straight chain, C3-C30 for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen on one or more carbons of the hydrocarbon backbone. Such substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF3, —CN, and the like.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF3, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and include both unsubstituted and substituted amines, e.g., a moiety that may be represented by the general formulas:

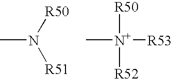

wherein R50, R51 and R52 each independently represent a hydrogen, an alkyl, an alkenyl, —(CH2)m-R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH2)m-R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

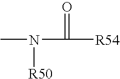

wherein R50 is as defined above, and R54 represents a hydrogen, an alkyl, an alkenyl or —(CH2)m-R61, where m and R61 are as defined above.

The term “amido” is art recognized as an amino-substituted carbonyl and includes a moiety that may be represented by the general formula:

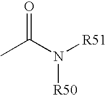

wherein R50 and R51 are as defined above. Certain embodiments of the amide in the present invention will not include imides which may be unstable.

The term “alkylthio” is art recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH2)m-R61, wherein m and R61 are defined above. Representative alkylthio groups include methylthio, ethyl thio, and the like.

The term “carbonyl” is art recognized and includes such moieties as may be represented by the general formulas:

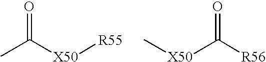

wherein X50 is a bond or represents an oxygen or a sulfur, and R55 represents a hydrogen, an alkyl, an alkenyl, —(CH2)m-R61 or a pharmaceutically acceptable salt, R56 represents a hydrogen, an alkyl, an alkenyl or —(CH2)m-R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH2)m-R61, where m and R61 are described above.

The term “sulfonate” is art recognized and includes a moiety that may be represented by the general formula:

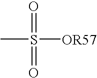

in which R57 is an electron pair, hydrogen, alkyl, cycloalkyl, or aryl.

The term “sulfate” is art recognized and includes a moiety that may be represented by the general formula:

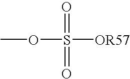

in which R57 is as defined above.

The term “sulfonamido” is art recognized and includes a moiety that may be represented by the general formula:

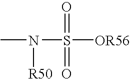

in which R50 and R56 are as defined above.

The term “sulfamoyl” is art-recognized and includes a moiety that may be represented by the general formula:

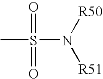

in which R50 and R51 are as defined above.

The term “sulfonyl” is art recognized and includes a moiety that may be represented by the general formula:

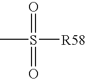

in which R58 is one of the following: hydrogen, alkyl, alkenyl, alkynyl, cycloalkyl, heterocyclyl, aryl or heteroaryl.

The term “sulfoxido” is art recognized and includes a moiety that may be represented by the general formula:

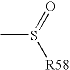

in which R58 is defined above.

The term “phosphoramidite” is art recognized and includes moieties represented by the general formulas:

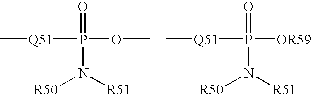

wherein Q51, R50, R51 and R59 are as defined above.

The term “phosphonamidite” is art recognized and includes moieties represented by the general formulas:

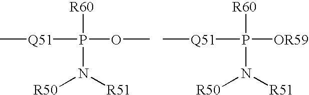

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

The term “selenoalkyl” is art recognized and includes an alkyl group having a substituted seleno group attached thereto. Exemplary “selenoethers” which may be substituted on the allyl are selected from one of —Se-alkyl, —Se-alkenyl, —Se-alkynyl, and —Se—(CH2)m-R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain monomeric subunits of the present invention may exist in particular geometric or stereoisomeric forms. In addition, polymers and other compositions of the present invention may also be optically active. The present invention contemplates all such compounds, including cis- and trans-isomers, R- and S-enantiomers, diastereomers, (d)-isomers, (I)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art recognized and includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2nd ed., Wiley, New York, (1991).

The phrase “hydroxyl-protecting group” is art recognized and includes those groups intended to protect a hydroxyl group against undesirable reactions during synthetic procedures and includes, for example, benzyl or other suitable esters or ethers groups known in the art.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (a) constant. This well known constant is described in many references, for instance, March, Advanced Organic Chemistry 251-59, McGraw Hill Book Company, New York, (1977). The Hammett constant values are generally negative for electron donating groups (σ(P)=−0.66 for NH2) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

Contemplated equivalents of the polymers, subunits and other compositions described herein include such materials which otherwise correspond thereto, and which have the same general properties thereof (e.g., biocompatible, nitric oxide generating), wherein one or more simple variations of substituents are made which do not adversely affect the efficacy of such molecule to achieve its intended purpose. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

Exemplary Nitric Oxide Agents

Nitric oxide agents contemplated by this disclosure include both nitric oxide generating agents and nitric oxide donating, or releasing, agents. Practitioners of the art will readily appreciate the circumstances under which various nitric oxide agents are appropriate for use in the aspects disclosed herein. For example, as described in the Exemplification section below, certain diazeniumdiolates and copper-ligand complexes generates and effective amount of nitric oxide that promotes angiogenesis in a patient in need thereof.

Nitric oxide generating agents are defined herein to include only those agents that do not have covalently attached nitric oxide releasing moieties, rather, nitric oxide generating agents are capable of generating nitric oxide when in contact with nitrosothiols, such as those found in bodily fluids such as blood.

For example, nitric oxide generating agents include metal-ligand complexes. For example, metal-ligand complexes include complexes that have a neutral carrier type ligand with a high metal binding affinity. In some embodiments, such ligands have a high binding affinity for copper. Metal-ligand complexes may have a planar square-type geometry that may provide a minimum amount of steric hindrance to the approach of an electron source to the center metal of the complex so that the metal ion can easily be reduced. Non-limitative examples of such metal-ligand complexes include nitrogen or sulfur donating compounds, such as Ne-donor macrocyclic ligands (x=2, 3, 4, 5, 6, 7, 8) such as cyclen, cyclam and their derivatives, and crown ethers and S_{y}-donor macrocycle-type ligands (y=2, 3, 4, 5, 6, 7, 8). In an embodiment, the metal-ligand macrocycle is a N₄ macrocycle.

Examples of a metal-cyclen complex includes those metal complexes that include the structure:

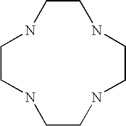

and derivatives of such cyclen ligands. Metal-cyclam structures can include structures such as:

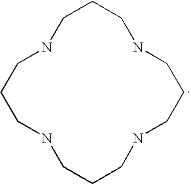

For example, metal-cyclam structures include 1,8 bis(pyridylmethyl)cyclam, 1,11-bis(pyridylmethyl)cyclam, and diooxocyclam ligands and structural isomers t Exemplary ligands include dibenzo[e,k]-2,3,8,9-tetraphenyl-1,4,7,10-tetraaza-cyclododeca-1,3,7,9-tetraene; dibenzo[e,k]-2,3,8,9-tetramethyl-1,4,7,10-tetraaza-cyclododeca-1,3,7,9-tetraene; dibenzo[e,k]-2,3,8,9-tetraethyl-1,4,7,10-tetraaza-cyclododeca-1,3,7,9-tetraene, and/or salts thereof. Such ligands can be modified to include halogen atoms.

A metal associated with a ligand includes metals and/or metal ions, for example, calcium, magnesium, cobalt, copper, manganese, iron, molybdenum, tungsten, vanadium, aluminum, chromium, zinc, nickel, platinum, tin, ions thereof, and/or mixtures thereof. In some embodiments, the metal entity in a metal-ligand complex may be associated with a ligand within the ligand or outside the ligand. A metal-ligand complex can be formed initially or can be formed once a ligand is placed in metal containing tissue or bodily fluids such as blood.

Other non-limiting examples of nitric oxide generating agents include, in general, enzymes having nitrate, nitrite, nitrosothiol reductase activity, for example, xanthine oxidase and nitrite and/or nitrate reductases derived from plants or bacteria. Nitric oxide generating agents may include hydrogel metal complexes. In an alternate embodiment, the nitric oxide generating agent may be metals and/or metal ions, for example, calcium, magnesium, cobalt, manganese, iron, molybdenum, tungsten, vanadium, aluminum, chromium, zinc, nickel, platinum, tin, ions thereof, and/or mixtures thereof. Nitric oxide generating agents may include copper (II) phosphate and various copper salts. In some embodiments, the metal entity in a metal-ligand complex may be associated with a ligand either, for example within the ligand or outside the ligand.

Without being limited to any theory, nitric oxide generating agents, when exposed to endogenous or exogenous sources of nitrates, nitrites, or nitrosothiols, and optionally in the presence of reducing agents, generates an active metal (for example, with coordination(I)) species that generates NO within or at the surface of a composition. It is to be understood that the sources of nitrates, nitrites, nitrosothiols and reducing agents may be from bodily fluids such as blood, within the composition, within the sensor, and/or may be injected intravenously or otherwise added or administered to the bodily fluid of interest.

The nitric oxide generating agents contemplated herein may decompose at a temperature that is higher than a typical processing temperature for the manufacture of analyte sensors, and/or at a higher temperature than a nitric oxide releasing agent. For example, the nitric oxide generating agents may decompose at a temperature above about 100° C., or even above about 125° C. In an embodiment, the nitric oxide generating agents contemplated by this disclosure are thermally stable.

Nitric oxide releasing agents are defined herein to include agents that have nitric oxide donor moieties covalently attached or otherwise bonded to the agent. Non-limiting examples of nitric oxide releasing agents include such agents as S-nitrosothiols, S-nitroso amino acids, S-nitroso-polypeptides, and nitrosoamines. One group of such nitric oxide donor moieties include the S-nitrosothiols, which are compounds that include at least one —S—NO group. Such compounds include S-nitroso-polypeptides (the term “polypeptide” includes proteins and also polyamino acids that do not possess an ascertained biological function, and derivatives thereof); S-nitrosylated amino acids (including natural and synthetic amino acids and their stereoisomers and racemic mixtures and derivatives thereof); S-nitrosated sugars, S-nitrosated-modified and unmodified oligonucleotides; and an S-nitrosated hydrocarbon where the hydrocarbon can be a branched or unbranched, and saturated or unsaturated aliphatic hydrocarbon, or an aromatic hydrocarbon; S-nitroso hydrocarbons having one or more substituent groups in addition to the S-nitroso group; and heterocyclic compounds. S-nitrosylated proteins include thiol-containing proteins (where the NO group is attached to one or more sulfur group on an amino acid or amino acid derivative thereof) from various functional classes including enzymes, such as tissue-type plasminogen activator (TPA) and cathepsin B; transport proteins, such as lipoproteins, heme proteins such as hemoglobin and serum albumin; and biologically protective proteins, such as the immunoglobulins and the cytokines. Other suitable S-nitrosothiols that are S-nitroso-angiotensin converting enzyme inhibitors.

Nitric oxide donor agents include compounds that include at least one —O—NO group. Such compounds include O-nitroso-polypeptides (the term “polypeptide” includes proteins and also polyamino acids that do not possess an ascertained biological function, and derivatives thereof); O-nitrosylated amino acids (including natural and synthetic amino acids and their stereoisomers and racemic mixtures and derivatives thereof); O-nitrosated sugars; O-nitrosated-modified and unmodified oligonucleotides; and an O-nitrosated hydrocarbon where the hydrocarbon can be a branched or unbranched, saturated or unsaturated aliphatic hydrocarbon, or an aromatic hydrocarbon; O-nitroso hydrocarbons having one or more substituent groups in addition to the O-nitroso group; and heterocyclic compounds.

Further nitric oxide donor agents include nitrites which have an—O—NO group wherein R is a protein, polypeptide, amino acid, branched or unbranched and saturated or unsaturated alkyl, aryl or a heterocyclic. N-nitrosoamines, which are compounds that include at least one —N—NO group, C-nitroso compounds that include at least one —C—NO, and compounds that include at least one —O—NO2 group.

Also contemplated by this disclosure as nitric oxide releasing/donor agents are diazeniumdiolates, such as those represented by:

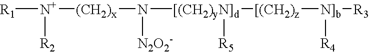

where d and b are independently selected from 0 or 1; R₁, R₂, R₃, R₄, R₅ are independently selected from hydrogen, C₃₋₈ cycloalkyl, C₁₋₁₂ straight or branched chain alkyl, benzyl, benzoyl, phthaloyl, acetyl, trifluoroacetal, p-tolyl, t-butooxycarbonyl, or 2,2,2-trichloro-t-butoxycarbonyl; z, x, and z are independently selected from an integer between 2 and 13 inclusive; and salts thereof.

Diazeniumdiolates include dialkyldiamine diazeniumdiolates such as compounds with the structure RN[N(O)NO]—(CH₂)₆NH₂+R, where R may be, for example, CH₃, CH₂CH₃, (CH₂)₂CH₃, (CH₂)₃CH₃, (CH₂)₄(CH₃), (CH₂)₅CH₃, and (CH₂)₁₁CH₃. In general, as the R chain varies in length, e.g. from R═CH₃ to (CH₂)₁₁CH₃, diazeniumdiolate increases in lipophilicity. For example, in some embodiments, a lipophilic diazeniumdiolate, such as (Z)-1-{N-Butyl-N [6-(N-butylammoniohexyl)amino]}-diazen-1-ium-1,2-diolate may be used so that, for example, leaching from a polymer composition or matrix is minimized.

### Compositions

For use in treating and/or preventing a variety of diseases, such as producing both anti-cell proliferative and anti-thrombotic effects, and/or promoting angiogenesis, and/or promoting the formation of blood vessel formation in hypoxic or ischemic tissue, such nitric oxide agents can be administered alone, or in a composition that is administered alone, or in and/or on a coating that at least is partially associated with a medical device. A variety of polymers may be used in such compositions.

A polymer for use in this invention may be biocompatible. Further, both non-biodegradable and biodegradable polymers may be used in the subject invention. As discussed below, the choice of polymer will depend in part on a variety of physical and chemical characteristics of such polymer and the use to which such polymer may be put.

Representative natural polymers include proteins, such as zein, modified zein, casein, gelatin, gluten, serum albumin, or collagen, and polysaccharides, such as cellulose, dextrans, hyaluronic acid, and polymers of alginic acid.

Representative synthetic polymers include polyphosphazines, poly(vinyl alcohols), polyamides, polycarbonates, polyalkylenes, polyacrylamides, polyanhydrides, poly(phosphoesters), polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyphosphates, polyesters, and polyurethanes. For example, polymers may include polydimethylsiloxane, ethylene vinyl acetate, nylons, polyacrylics, polymethyl methacrylate including those with alkyl chain lengths from about 2 to about 8 carbons and/or with a molecular weight of about 50,000 to about 9,000,000, polyethylenes, polypropylenes, polystyrenes, poly(vinyl chloride) (PVC), and polytetrafluoroethylene (PTFE). One or more polymers may be used in combination, for example, poly(butylmethacrylate) and poly(ethylene-co-vinyl acetate). Silicon rubbers may also be used as a polymer.

Synthetically modified natural polymers include alkyl celluloses, hydroxyalkyl celluloses, cellulose ethers, cellulose esters, and nitrocelluloses. Other like polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

All of the subject polymers may be provided as copolymers or terpolymers. These polymers may be obtained from chemical suppliers or else synthesized from monomers obtained from these suppliers using standard techniques.

In certain embodiments, the polymers are comprised almost entirely, if not entirely, of the same subunit. Alternatively, in other embodiments, the polymers may be copolymers, in which different subunits and/or other monomeric units are incorporated into the polymer. In certain instances, the polymers are random copolymers, in which the different subunits and/or other monomeric units are distributed randomly throughout the polymer chain.

In other embodiments, the different types of monomeric units, be they one or more subunits depicted by the subject formulas or other monomeric units, are distributed randomly throughout the chain. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing or treatment.

In certain embodiments, the subject polymers may be cross-linked. For example, substituents of the polymeric chain, may be selected to permit additional inter-chain cross-linking by covalent or electrostatic (including hydrogen-binding or the formation of salt bridges), e.g., by the use of a organic residue appropriately substituted.

The ratio of different subunits in any polymer as described above may vary. For example, in certain embodiments, polymers may be composed almost entirely, if not entirely, of a single monomeric element. Alternatively, in other instances, the polymers are effectively composed of two different subunits, in which the percentage of each subunit may vary from less than 1:99 to more than 99:1, or alternatively 10:90, 15:85, 25:75, 40:60, 50:50, 60:40, 75:25, 85:15, 90:10 or the like. In other embodiments, in which three or more different monomeric units are present, the present invention contemplates a range of mixtures like those taught for the two-component systems.

In certain embodiments, the polymeric chains of the subject compositions, e.g., which include repetitive elements shown in any of the subject formulas, have average molecular weights ranging from about 2000 or less to about 10,000,000 or more. Number-average molecular weight (Mn) may also vary widely, but generally fall in the range of about 1,000 to about 10,000,000. Within a given sample of a subject polymer, a wide range of molecular weights may be present. For example, molecules within the sample may have molecular weights which differ by a factor of 2, 5, 10, 20, 50, 100, or more, or which differ from the average molecular weight by a factor of 2, 5, 10, 20, 50, 100, or more.

One method to determine molecular weight is by gel permeation chromatography (“GPC”), e.g., mixed bed columns, CH₂Cl₂ solvent, light scattering detector, and off-line dn/dc. Other methods are known in the art.

In other embodiments, the polymer composition of the invention may be a flexible or flowable material. When the polymer used is itself flowable, the polymer composition of the invention, even when viscous, need not include a biocompatible solvent to be flowable, although trace or residual amounts of biocompatible solvents may still be present.

In certain embodiments, a fluid polymer may be especially suitable for the treatment of a patient with wounds or a patient in need of rapid healing or endothelialization of intravascular luminal surfaces. A fluid material may be adapted for injection or instillation into a tissue mass or into an actual or potential space. Certain types of fluid polymers may be termed flowable. A flowable material, often capable of assuming the shape of the contours of an irregular space, may be delivered to a portion of an actual or potential space to flow therefrom into a larger portion of the space. In this way, the flowable material may come to coat an entire post-operative surgical site after being inserted through an edge of an incision or after being instilled through a drain or catheter left in the surgical bed. Alternatively, if the flowable material is inserted under pressure through a device such as a needle or a catheter, it may perform hydrodissection, thus opening up a potential space and simultaneously coating the space with polymer. Such potential spaces suitable for hydrodissection may be found in various identifiable anatomic areas where, for example, wounds or areas may require treatment, for example, endothelialization of intrvascular luminal surfaces. A flowable polymer may be particularly adapted for instillation through a needle, catheter or other delivery device such as an endoscope, since its flowable characteristics allow it to reach surfaces that extend beyond the immediate reach of the delivery device. A flowable polymer in a highly fluid state may be suitable for injection through needles or catheters into tissue masses, such as wound sites, and, for example, can be injection directly into or on tissue, or can be administered by intravascular perfusion or infusion. Physical properties of polymers may be adjusted to achieve a desirable state of fluidity or flowability by modification of their chemical components and crosslinking, using methods familiar to practitioners of ordinary skill in the art.

A flexible polymer may be used in the fabrication or as full or partial coating of a solid article, or as a delivery system itself. Flexibility involves having the capacity to be repeatedly bent and restored to its original shape. Solid articles made from, or at least partially coated with, flexible polymers are adapted for placement in anatomic areas where they will encounter the motion of adjacent organs or body walls. Certain areas of motion are familiar to practitioners dealing with implantable devices, such as for example, stents. A flexible solid article can thus be sufficiently deformed by those moving tissues that it does not cause tissue damage. Flexibility is particularly advantageous where a solid article might be dislodged from its original position and thereby encounter an unanticipated moving structure; flexibility may allow the solid article to bend out of the way of the moving structure instead of injuring it. Such a flexible article might be suitable for inserting into pulsatile vessels such as the internal carotid artery, the cerebral arteries, the middle meningeal artery, the basilar artery, the vertebral artery, and the spinal arteries, or for inserting into more delicate structures in the bead such as the venous sinuses that may also be affected by local movements. Use of a solid article or device according to the present invention in the aforesaid ways may allow less extensive dissections to be carried out with surgical preservation and protection of structures important to function. Solid articles may be configured as three-dimensional structures suitable for implantation in specific anatomic areas. For example, a solid article, such as a stent, or a solid article formed from a polymer may be implanted intravenously, subcutaneously, or may be implantable into the margins of a resected bone or cartilaginous structure and may be fabricated according to the present invention to carry a nitric oxide agent. Solid articles may be formed as films, meshes, sheets, tubes, or any other shape appropriate to the dimensions and functional requirements of the particular anatomic area. Physical properties of polymers may be adjusted to attain a desirable degree of flexibility by modification of the chemical components and crosslinking thereof, using methods familiar to practitioners of ordinary skill in the art.

In certain embodiments, the subject polymers are soluble in one or more common organic solvents for ease of fabrication and processing. Common organic solvents include such solvents as chloroform, dichloromethane, dichloroethane, 2-butanone, butyl acetate, ethyl butyrate, acetone, and ethyl acetate.

Polymers that resist protein adsorption may also be used in compositions contemplated by this disclosure. Such polymers include polyethylene glycols, polyurethanes and silicone elastomer, silica containing polymers, and poly(vinyl)chlorides. Other polymers that may be used include tecophilic polyurethanes, PDMS co-polymers, carbamates, and the like.

The mechanical properties of the polymer may be important for the processability of making molded or pressed articles for implantation or for use as a coating or layer on a medical device. For example, the glass transition temperature may vary widely but must be sufficiently lower than the temperature of decomposition to accommodate conventional fabrication techniques, such as compression molding, extrusion or injection molding.

The polymer and/or polymer(s) may be selected so that when used as a part of a composition for coating a medical device, the physical characteristics of the coating, for example, durability, flexibility, and/or expandibility will be adequate for use on or within a medical device.

Nitric oxide agents may be incorporated within a polymer composition or on the surface of a polymer composition, or both. In some embodiments, the nitric oxide agents are at least partially bonded, covalently or otherwise, to a polymer composition. In another embodiment, the nitric oxide agents may be bonded directly or indirectly to a metal surface, for example, the metal surface of a medical device. Such compositions may generate nitric oxide at physiologically relevant levels of NO from physiological levels of various RSNO species (e.g., greater than or equal to about 0.5×10-10 mol-cm-2 min-1) for extended periods (for example, about 2 weeks or more).

Such nitric oxide agent compositions may further include other therapeutic agents, including for example: anti-thrombotic agents such as heparin, heparin derivatives, urokinase, and pack (dextrophenylalanine proline arginine chloromethylketone); anti-inflammatory agents such as dexamethasone, prednisolone, corticosterone, budesonide, estrogen, sulfasalazine and mesalamine; antineoplastic/antiproliferative/anti-miotic agents such as paclitaxel, 5-fluorouracil, cisplatin, vinblastine, vincristine, rapamycin, epothilones, endostatin, angiostatin, angiopeptin, monoclonal antibodies capable of blocking smooth muscle cell proliferation, and thymidine kinase inhibitors; anesthetic agents such as lidocaine, bupivacaine and ropivacaine; anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; vascular cell growth promoters such as growth factors, including platelet-derived growth factor, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); prostacyclin analogs; cholesterol-lowering agents; angiopoietins; antimicrobial agents such as triclosan, cephalosporins, aminoglycosides and nitrofurantoin; cytotoxic agents, cytostatic agents and cell proliferation affectors; vasodilating agents; and agents that interfere with endogenous vascoactive mechanisms. Other agents that may be included include Ca-channel blockers including: benzothiazapines such as diltiazem and clentiazem, dihydropyridines such as nifedipine, amlodipine and nicardapine, phenylalkylamines such as verapamil, serotonin pathway modulators including: 5-HT antagonists such as ketanserin and naftidrofuryl, 5-HT uptake inhibitors such as fluoxetine, cyclic nucleotide pathway agents including: phosphodiesterase inhibitors such as cilostazole and dipyridamole, adenylate/guanylate cyclase stimulants such as forskolin, adenosine analogs, catecholamine modulators including: α-antagonists such as prazosin and bunazosine, α-antagonists such as propranolol, α/β-antagonists such as labetalol and carvedilol, endothelin receptor antagonists, ACE inhibitors such as cilazapril, fosinopril and enalapril, ATII-receptor antagonists such as saralasin and losartin, platelet adhesion inhibitors such as albumin and polyethylene oxide, platelet aggregation inhibitors including, aspirin and thienopyridine (ticlopidine, clopidogrel), GP IIb/IIIa inhibitors such as abciximab, epitifibatide and tirofiban, coagulation pathway modulators including heparinoids such as heparin, low molecular weight heparin, dextran sulfate and β-cyclodextrin tetradecasulfate, FXa inhibitors such as antistatin and TAP (tick anticoagulant peptide), vitamin K inhibitors such as warfarin, activated protein C, cyclooxygenase pathway inhibitors such as aspirin, ibuprofen, flurbiprofen, indomethacin and sulfinpyrazone, Natural and synthetic corticosteroids such as dexamethasone, prednisolone, methprednisolone and hydrocortisone, Lipoxygenase pathway inhibitors such as nordihydroguairetic acid and caffeic acid, Leukotriene receptor antagonists, Antagonists of E- and P-selectins, inhibitors of VCAM-1 and ICAM-1 interactions, prostaglandins and analogs thereof including, Prostaglandins such as PGE1 and PGI2, prostacylcin analogs such as ciprostene, epoprostenol, carbacyclin, iloprost and beraprost, macrophage activation preventers including bisphosphonates, HMG-CoA reductase inhibitors such as lovastatin, pravastatin, fluvastatin, simvastatin and cerivastatin, free-radical scavengers/antioxidants such as probucol, vitamins C and E, ebselen, trans-retinoic acid and SOD mimics, agents affecting various growth factors including, FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-α antibodies, EGF pathway agents such as EGF antibodies, receptor antagonists and chimeric fusion proteins, thromboxane A2 (TXA2) pathway modulators such as sulotroban, vapiprost, dazoxiben and ridogrel, MMP pathway inhibitors such as marimastat, ilomastat and metastat, antiproliferative/antineoplastic agents including antimetabolites such as purine analogs (e.g., 6-mercaptopurine, thioguanine), pyrimidine analogs (e.g., cytarabine and 5-fluorouracil) and methotrexate, nitrogen mustards, alkyl sulfonates, ethylenimines, and antibiotics (e.g., daunorubicin, doxorubicin).

Exemplary genetic therapeutic agents may also be included in the contemplated compositions. Genetic therapeutic agents include: anti-sense DNA and RNA; DNA coding for: anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β platelet-derived endothelial growth factor, platelet-derived growth factor, tumor necrosis factor α, hepatocyte growth factor and insulin like growth factor, cell cycle inhibitors including CD inhibitors, thymidine kinase (“TK”) and other agents useful for interfering with cell proliferation, and the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them. Vectors of interest for delivery of genetic therapeutic agents include Plasmids Viral vectors such as adenovirus (AV), adenoassociated virus (AAV) and lentivirus. Non-viral vectors can be included such as lipids, liposomes and cationic lipids.

### Devices and Systems

A patient in need of in need of an insertable medical device may also be in need of, for example, treatment for promoting or initiating angiogenesis. A medical device may include a nitric oxide agent, or a composition including a nitric oxide agent.

Such medical devices include, for example, an intravascular medical or delivery device, such as vascular catheters, (e.g. balloon catheter, an injection catheter, and an infusion catheter), a stent, a stent graft, vascular grafts, guide wires, balloons, filters (for example, vena cava filters), aneurysm fillers (including for example Guglielmi detachable coils), intraluminal paving systems, urinary catheters, valves, stets, shunts, pacemaker leads, implantable defibrillator, adventitial wrap, or a distal protection device. Other medical articles for the delivery of a nitric oxide agent for use in the promotion of, for example blood vessel formation in a patient in need thereof include patches and bandages.

The nitric oxide agents associated with a drug delivery system or device can be, for example, provided within a polymer matrix, dissolved or dispersed in a solution, or adsorbed or coated at least partially on a tissue-contacting or fluid-contacting surface of a medical article, such a metallic medical device.

Arterial stents and other medical devices, for example, may be fabricated from a variety of compounds including surgical grade metals, metallic alloys and biocompatible synthetic polymers such as polypropylene, polyethylene, polyesters, polyethers, polyurethanes and polylactides. Such devices may include polymeric coatings that include nitric oxide agents, such as the compositions disclosed herein. Alternatively, nitric oxide agents may be coupled directly to a metallic surface of a device through chemical intermediates, for example, silane intermediates.

The metallic medical devices of the present invention include, but are not limited to, arterial stents, guide wires, catheters, trocar needles, bone anchors, bone screws, protective platings, hip and joint implants, electrical leads, biosensors and probes. Suitable metallic surfaces for coating include, but are not limited to, stainless steel, nickel, titanium, aluminum, copper, gold, silver, platinum and combinations thereof.

For example, a nitric oxide agent and polymer can be combined, to form a composition of this disclosure, in any fashion known to those skilled in the art, and, if necessary, combined with a solvent to create a solution, and then applied to the surface of the medical device using methods known to those skilled in the art including, but not limited to, dipping, spraying, brushing, imbibing and rolling.

Biocompatible compositions and devices and articles thereof, may be prepared in a variety of ways known in the art. A polymer composition of this disclosure may be melt processed using conventional extrusion or injection molding techniques, or these products may be prepared by dissolving in an appropriate solvent, followed by formation of the device, and subsequent removal of the solvent by evaporation or extraction, e.g., by spray drying. By these methods, the polymers may be formed into articles of almost any size or shape desired, for example, implantable solid discs or wafers or injectable or insertable rods, needles, or formed as at least a partial coating on a medical device. Typical medical articles also include such as implants such as laminates for degradable fabric or coatings to be placed within the body, e.g. at a wound site, or on other implant devices.

When the polymer composition of the invention is flexible or flowable, it may be placed anywhere within the body. In certain embodiments, a polymer composition according to the present invention may also be incorporated in or on an access devices so that nitric oxide agent is in contact with anatomic area or fluid within which the access device resides, thereby enhancing or promoting, for example, blood perfusion or angiogenesis.

The nitric oxide agents of the present invention are used in amounts that are therapeutically effective, which varies widely depending largely on the particular nitric oxide agent being used. The amount of nitric oxide agent incorporated into the composition also depends upon the desired release profile of nitric oxide, the concentration of the agent required for a biological effect, and the length of time that the biologically active substance has to be released for treatment. In certain embodiments, the biologically active substance may be blended with the polymer matrix of the invention at different loading levels, preferably at room temperature and without the need for an organic solvent. In other embodiments, the compositions of the present invention may be formulated as microspheres, hydropolymers such as hydrogels, gels, or sprays.

For delivery of an nitric oxide agent or some other biologically active substance, the agent or substance is added to the polymer composition. A variety of methods are known in the art for encapsulating a biologically active substance in a polymer. For example, the agent or substance may be dissolved to form a homogeneous solution of reasonably constant concentration in the polymer composition, or it may be dispersed to form a suspension or dispersion within the polymer composition at a desired level of “loading” (grams of biologically active substance per grams of total composition including the biologically active substance, usually expressed as a percentage).

### Administration and Methods

For nitric oxide to effectively activate, for example, angiogenesis, nitric oxide agents may be administered to, for example, an injured vessel wall as soon as possible after the initial insult. It may be necessary, in some embodiments, to maintain administration of such nitric oxide agents for sustained periods such as at least 1 day, 1 week or up to or over 2 weeks. An arterial stent, for example, that includes such an agent may, in one embodiment, allow for delivery for the sustained, localized delivery of NO, thereby promoting angiogenesis.

In some embodiments, stent coatings that include the disclosed compositions, for example, are capable of NO generation for at least 1 week, 2 week, or even 4 weeks. In some embodiments, prolonged release of several months or even longer is possible.

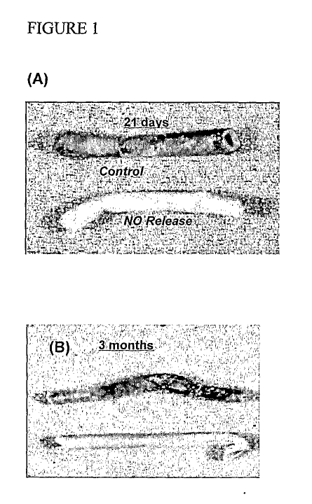

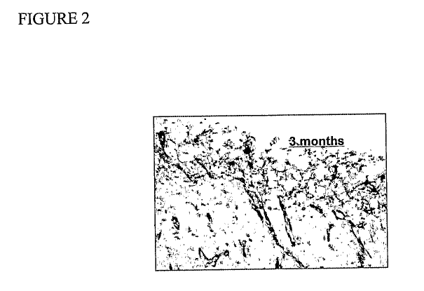

In some embodiments, nitric oxide release or generation from nitric oxide agents may cease after about 3 weeks. In a further embodiment, there may be a lasting effect on preventing significant thrombus formation on the inner wall of a medical device, such as a stent, after implant periods of about 3 months even after, for example, an implanted device has ceased generating or releasing nitric oxide. Similarly, FIGS. 1 and 2 shows that fibrotic encapsulation on the outside of such grafts is also suppressed at the 3 month time point, even though NO release had stopped 2+ months earlier. These results suggest that if the initial inflammatory response of the surrounding tissue cells can be influenced by the agent released from the surface of the implant, and normal wound healing can be promoted without recruitment of neutrophils and other inflammatory cells, there is the great potential for a long lasting benefit even in the absence of long-term release of the therapeutic agent.

Certain exemplary treatment methods and indications for various aspects of enhancing blood perfusion, promoting blood vessel formation, promoting angiogenesis, promoting wound healing, and/or preventing incorporation and/or tissue encapsulation of medical devices are described below. It is understood, however, that these descriptions are intended as illustrative only, not intended to be limiting in any way, and that other modifications and variations of these illustrative embodiments may be contemplated without departing from the scope of the present invention.

In some embodiments, a method is provided for a patients in need thereof, for promoting angiogenenic effects such as enhancing vascularization/blood flow to ischemic cells/tissues, for example, for promoting angiogenesis when coronary artery disease, e.g. ischemic myocardium, myocardial infarction, ischemic cardiomyopathy, or peripheral arterial disease, such as chronic limb ischemia claudication (skeletal muscle), rest pain/ischemic ulceration/gangrene is present or suspected. Treatment of an patient in need of promoting angiogenesis may be indicated in the event of for example, ischemic stroke/neuropathy, such as brain/nerve tissue, for example, ischemic pneumbra around stroke/infarct.

A method is also provided to promote healing and/or endothelialization of intravascular luminal surfaces in a patient in need thereof, for example, to promote endothelialization of unstable/ulcerated atherosclerotic plaque, for example in coronary/carotid arteries, or on de-endothelialized luminal surfaces such as those found following an endarterectomy, for example within the carotid artery, thrombectomy (either/or arterial/venous), angioplasty, such as balloon, laser, or cryogenic angioplasty, an atherectomy, or following thrombolysis, by administering a composition that includes a nitric oxide agent. Without being limited to any theory, nitric oxide may be final common mediator of VEGF-induced angiogenesis in-vivo.

Compositions provided herein may also assist in resolution of acute, or chronic arterial and/or venous thrombosis, for example revascularization and/or neovascularization and/or recanalization. In another embodiment, compositions are provided that promote development of neocapillary beds for gene therapy applications, organ regeneration applications, and for bioartificial hybrid organs (e.g. pancreas, kidney, lung, liver) placement. Methods are also provided to promote and/or enhance wound healing and/or for promoting granulation tissue, for example, for chronic wounds such as ischemic, diabetic, neuropathic, venous statis based wounds.

In one embodiment, the compositions disclosed herein may be used to prevent fibrous tissue formation after incisions, or to treat neointimal hyperplasia. For example, a method is provided herein of treating a site of vascular compromise to seal a puncture or opening, and to treat, suppress or prevent a tissue response at such site, by administering a composition of this disclosure. In an embodiment, the method may include administering a nitric oxide agent or composition including a nitric oxide agent, such as disclosed herein, or a nitric oxide agent and a hemostatic device or material, and applying, for example, the composition to the site.

Compositions comprising nitric oxide agents may also be used to prevent incorporation and/or tissue encapsulation of medical devices, or surfaces thereof, for example, artificial or natural replacement surfaces, for example, those placed in body cavities such as the thorax, abdomen, and/or hernia, devices such as implantable biosensors, for example intravascular, brain, heart, gut sensors, pacemakers/leads, implantable drug delivery systems, and other biomechanical devices/surfaces such as bioartificial organs, joints, or heart valves.

In some embodiments, implantable devices may include surface-specific engineering such that specific surfaces are designed to avoid incorporation encapsulation effect whereas other surfaces on or within the same device are designed to promote incorporation or encapsulation, angiogenesis or other agent-specific effects. For example, one or more surfaces of a device may include a first nitric oxide agent or composition that comprises a first nitric oxide agent and one or more different surfaces of the same device may include a second nitric oxide agent, or no nitric oxide agent, or composition that comprises a second nitric oxide agent, so that different surfaces have different qualities.

In some embodiments, a composition, such as a composition disclosed herein that is disposed at least partially on a medical device such as a stent, inhibits multiple pathways (both restenosis and thrombosis in addition to inflammation and infection) and causes minimal systemic affects. In some embodiments, a nitric oxide agent can be administered to, for example, reduce restenosis, thrombosis as well as inflammation and infection. Further, NO's lifetime in blood is very short (<1 see) owing to its rapid reaction with hemoglobin. Hence, the NO originating from, for example, a coating that comprises a disclosed composition may release/generate this radical species at levels comparable to the normal endothial cells and may be unlikely to have any systemic effects. Because NO is produced by the body continuously to maintain normal hemostasis, it is likely to provide a route to safely prevent thrombosis and smooth muscle cell proliferation that occur when, for example, stents are implanted, thus extending the lifetime and reducing the risks associated with such implants.

As contemplated by the present invention, the nitric oxide agent will release or generate nitric oxide from a subject composition in an amount sufficient to deliver to a patient a therapeutically effective amount of such agent as part of a prophylactic or therapeutic treatment.

For example, a pharmaceutical composition suitable for increasing angiogenesis and disposed on a medical device suitable for implantation in a patient is provided, wherein the composition comprises at least one of: a nitric-oxide releasing agent and nitric-oxide generating agent, wherein said composition increases angiogenesis by at least about 1%, 10%, about 20%, or even about 25% as compared with angiogenesis generated by a composition disposed on a medical device without said nitric-oxide releasing and nitric-oxide generating agent.

In certain aspects, the subject compositions, upon contact with body fluids including blood, lymph, tissue fluid or the like, release or generate nitric oxide over a sustained or extended period. Such a system may result in prolonged delivery (over, for example, 2 to 25 days) of effective amounts of nitric oxide. Such delivery may result in blood vessel formation over an even longer time, for example, even over 1 month, 2 months or 3 months in a site surrounding the composition. Such blood vessel formation may occur even after delivery of nitric oxide to a site has substantially ceased. A dosage form may be administered as is necessary depending on the subject being treated, the severity of the affliction, the judgment of the prescribing physician, and the like.

The efficacy of treatment with the subject compositions may be determined in a number of ways.

The efficacy of treatment using the subject compositions may be compared to treatment regimens known in art in which a nitric oxide agent is not within a treatment regimen. For example, treatment with a subject composition that comprises a nitric oxide agent is expected to result in fewer thrombotic and cell proliferative effects, than treatment with another agent. Alternatively, treatment with a subject composition results in an increase in blood vessel formation in sheep, and it is expected that the same will result in other mammals, and in particular humans.

Alternatively, the different treatment regimens may be analyzed by comparing the therapeutic index for each of them, with treatment with a subject composition as compared to another regimen having a therapeutic index two, three, five or seven times that of, or even one, two, three or more orders of magnitude greater than, treatment with another method using a different composition or nitric oxide agent.

### REFERENCES

All publications and patents mentioned herein, including those items listed below, are hereby incorporated by reference in their entirety as if each individual publication or patent was specifically and individually indicated to be incorporated by reference. In case of conflict, the present application, including any definitions herein, will control.
Fleser et al. Journal of Vascular Surgery 803-811 (October 2003); Batchelor et al., J. Med. Chem. v. 46 5153-5161 (2003).

### EXEMPLIFICATION

The invention now being generally described, it will be more readily understood by reference to the following examples which are included merely for purposes of illustration of certain aspects and embodiments of the present invention and are not intended to limit the invention in any way.

### Example 1

### Sheep Model

A total of 12 grafts were placed in six sheep in a blinded, randomized study: NO-releasing grafts (n=5 grafts); sham-coated control grafts (n=4 grafts); uncoated control grafts (n=3 grafts). Small-diameter polyurethane vascular grafts (5-mm internal diameter; 25-cm length; Vectra™ vascular access graft, Bard-Impra, Murray Hill. N.J.) were coated with a NO-releasing multi-layer PVC material containing the NO donor.

Vascular grafts were dip-coated to create multi-layer films of plasticized poly(vinyl chloride) on the inner surface. Control grafts were either uncoated or sham-coated grafts of equivalent size and length. Sham-coated control grafts were prepared with the same polymer layers used in the NO-releasing grafts, however the NO-releasing compound and anion additive were absent. Grafts were explanted after 21 d.

### Example 2

### Factor VIII

FIG. 2 illustrates a sample immunostained with Factor VIII. An endogenous peroxidase block is used with the sample, with 20 minutes of 0.3% H2O2, and then rinsed with PBS 3×. A 10% serum from species of secondary antibody (i.e. if secondary is goat anti-mouse, use goat serum) for 10 minutes is used. A Zymed CAS blocking reagent (universal blocking) may be used. A primary antibody is used for 30-90 minutes, and rinsed with PBS 3×-2 minutes. A secondary antibody is used for 30 minutes and then rinsed with PBS 3×-2 minutes, followed by an enzyme conjugate for 20-30 minutes, and rinse with PBS 3×-2 minutes. DAB, AEC Chromagen is used, and development of color is watched with microscope, around 2-5 minutes. Rinse with dH₂O. The sample is counterstained with hematoxylin. Coverslip with synthetic or aqueous mounting media as required for specific chromagen. Alternatively, an anti-VEGF antibody is used as specific to vascular endothelial cells.

### Example 3

The effect of NO on tissue incorporation was examined on the outer surface of the grafts following three week (six sheep) and three month (three sheep) implantation. FIG. 1 (A&B) shows representative explanted grafts at both time points. NO-releasing grafts showed reduced incorporation of the fibrotic capsule into the graft surface (abluminal) as compared to sham-coated, non-NO releasing grafts. Sections of the capsule surrounding the grafts were immunostained for Factor VIII and VEGF. FIG. 2 shows prominent Factor VIII staining in the area immediately surrounding the graft. Tissue farther up and downstream of the graft does not show this staining pattern, nor does the tissue around the control grafts. VEGF staining was also seen at the interface of the non-adherent tissue adjacent to the abluminal surface of the graft. The effect of NO on tissue incorporation is prolonged. For the three month implant studies, NO was released for only 21 d, yet the effect on tissue incorporation was still evident at 3 months.

### Example 4

### In-Vitro Angiogenesis Model

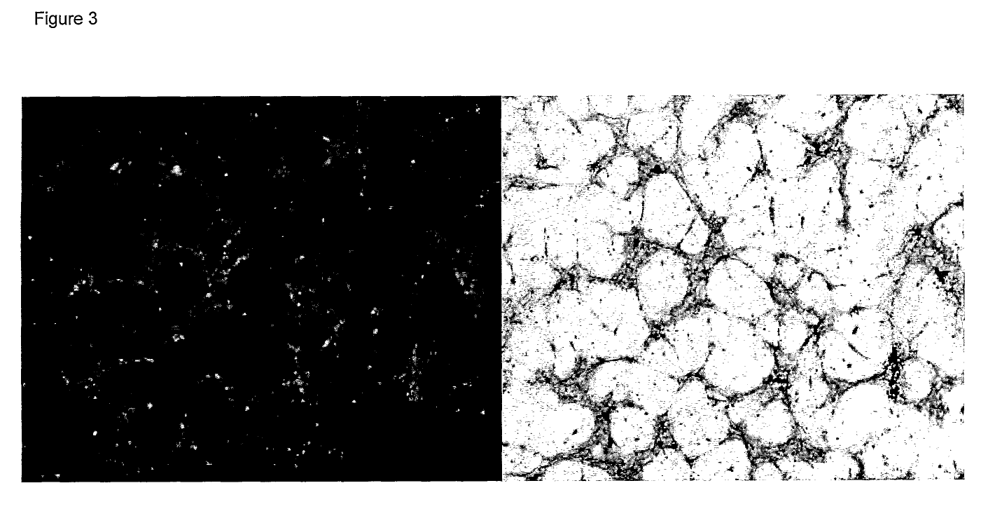

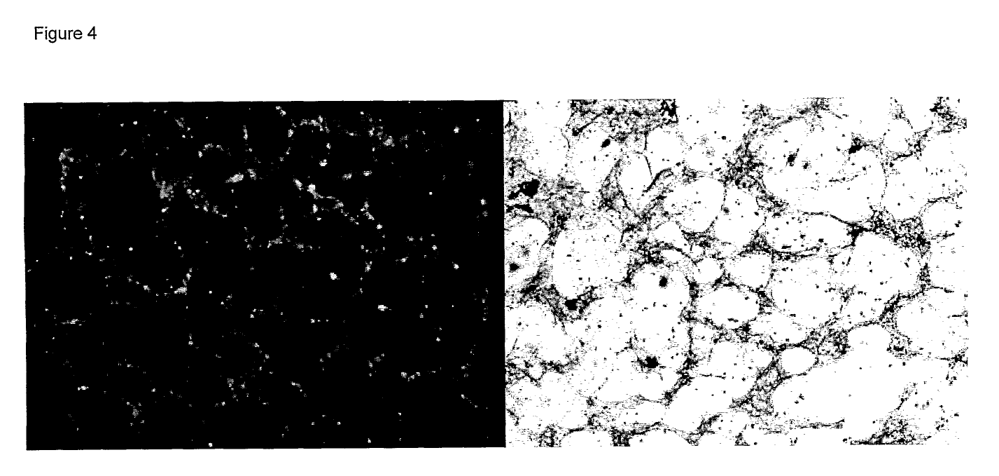

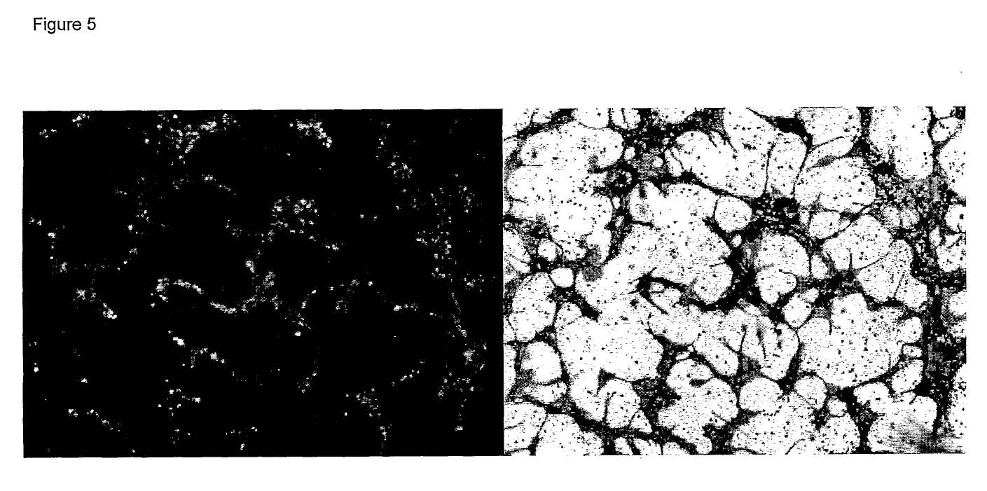

FIG. 3 shows network formation in HUVECs cultured in collagen gel matrix and pulsed with PMA, bFGF, and NOC18, a diazeniumdiolate, (10-9) for 2 hr followed by PMA, bFGF and VEGF-C for 70 hr. FIG. 4 depicts network formation in HUVECs cultured in collagen gel matrix and treated with PMA, bFGF, and VEGF-C for 72 hr (Positive control). FIG. 5 shows network formation in HUVECs cultured in collagen gel matrix and treated with PMA and bFGF, for 72 hr. Image on left of each figure is stained with phalloidin (cytoskeletal Actin-F marker) and the nuclear dye, Hoechst. Image on right depicts network formation (red lines) used by AngioQuant quantification program (Niemiströ et al 2005). Magnification=4×. The number of networks is 48 in FIG. 3, 44 in FIG. 4, 12 in FIG. 5.

### Example 5

### In-Vivo Angiogenesis Rabbit Implant Studies

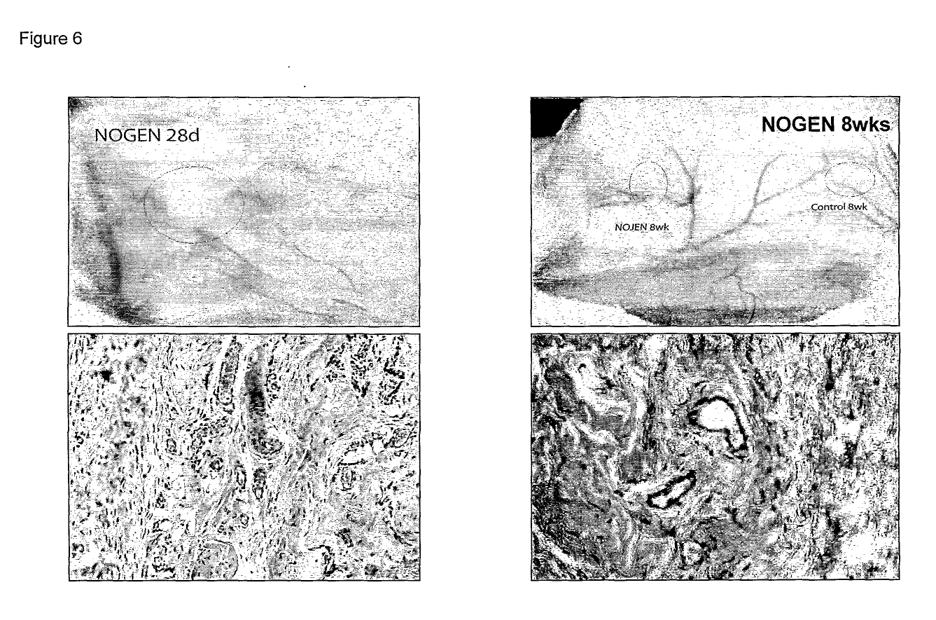

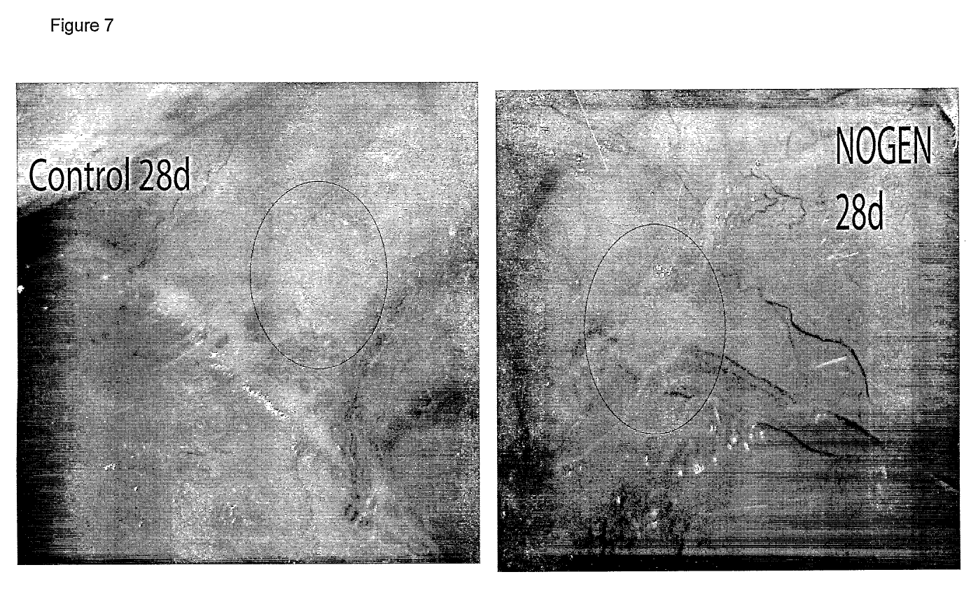

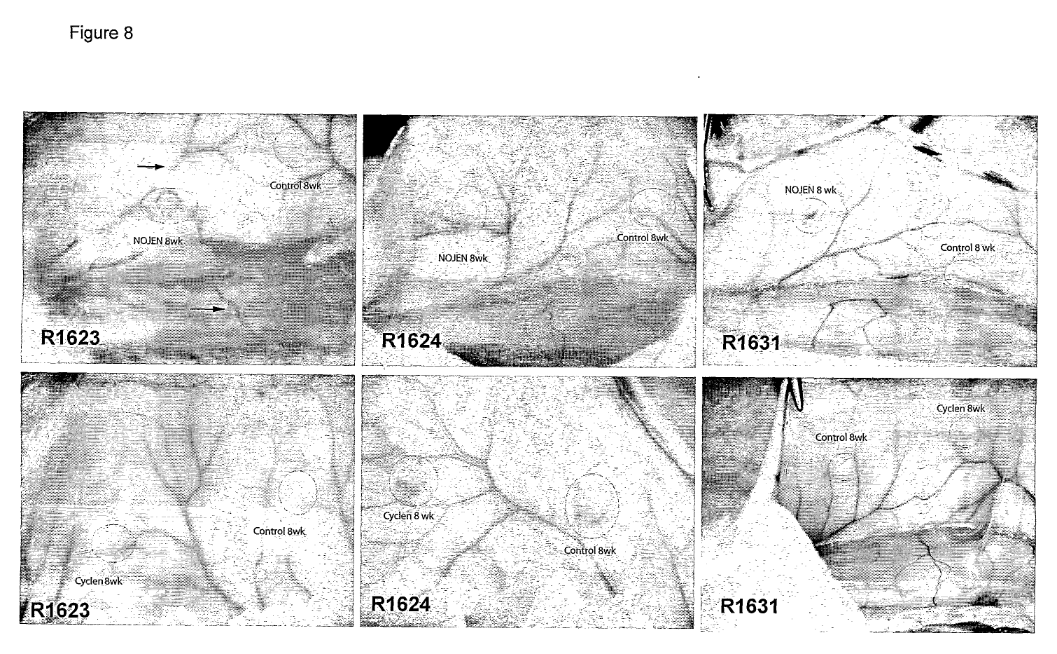

Rabbits were implanted subcutaneously with nitric oxide generating agent dibenzo[e,k]-2,3,8,9-tetramethyl-1,4,7,10-tetraaza-cyclododeca-1,3,7,9-tetraene, cyclen and plain polymer disks. FIG. 6 shoes nitric oxide agent explants at 28 d and 8 weeks (top row) showed increased angiogenic activity versus control (not shown). Explants were sectioned and stained with H&E. The bottom row shows increased blood vessel formation around nitric oxide agent disks 20× magnification. FIG. 7 shows control vs. nitric oxide generating agent at 28 d. FIG. 8 shows implants with nitric oxide generating agent, cyclen alone, and contl at 8 wk.

### EQUIVALENTS

Those skilled in the art will recognize, or will be able to ascertain using no more than routine experimentation, many equivalents to the specific embodiments and practices of the invention described herein. Such equivalents are intended to be encompassed by the following claims.

1. A method of promoting blood vessel formation in hypoxic or ischemic tissue in a patient in need thereof, comprising contacting said tissue with a biocompatible composition comprising a nitric oxide releasing agent or nitric oxide generating agent thereby generating an effective amount of nitric oxide to said tissue.

2. The method of claim 1, wherein said composition promotes blood vessel formation by promoting angiogenesis.

3. The method of claim 1, wherein said composition promotes blood vessel formation by promoting formation or maturation of collateral blood vessels.

4. The method of claim 1, wherein said tissue is cardiac tissue, neural tissue, muscle, skin, bone, or visceral organ tissue.

5. A method of promoting angiogenesis in a subject in need thereof, comprising implanting a biocompatible composition comprising a nitric oxide releasing agent or nitric oxide generating agent into said subject at a tissue locus thereby generating an effective amount of nitric oxide to said tissue.

6. The method of claim 5, wherein the nitric oxide releasing agent is a diazeniumdiolate.

7. The method of claim 5, wherein the nitric oxide generating agent is a metal-ligand complex capable of reducing nitrosothiol species to nitric oxide.

8. The method of claim 7, wherein the metal-ligand complex is a metal-cyclen complex or metal-cyclam complex.

9. The method of claim 7, wherein the metal-ligand complex is a copper-cyclen complex or a copper-cyclam complex.

10. The method of claim 7, wherein the metal-ligand complex is a N₄ donor type macrocycle.

11. The method of claim 5 wherein nitric oxide generated or released by said composition diffuses at least 10 microns, through tissue, away from a surface of said composition.

12. The method of claim 5, wherein nitric oxide generated or released by said composition diffuses at least 15, 20, 25, 35, 50, 75, or 100 microns, through tissue, away from a surface of said composition.

13. The method of claim 5, wherein said tissue locus is experiencing or at risk of insufficient blood perfusion.

14. The method of claim 5, wherein the composition further comprises a polymer having said nitric oxide releasing agent or nitric oxide generating agent dispersed therein or thereon.

15. The method of claim 14, wherein the composition further comprises a pro-angiogenic factor.

16. The method of claim 15, wherein said pro-angiogenic factor is vascular endothelial growth factor (VEGF), fibroblast growth factor (FGF), hepatocyte growth factor (HGF), platelet-derived growth factor (PDGF), interleukin 6 (IL-6), monocyte chemotactic protein 1 (MCP-1), granulocyte-macrophage colony stimulating factor (GM-CSF), or transforming growth factor β (TGFβ).

17. The method of claim 5, wherein the composition is a coating or film disposed on a surface of an implantable medical device.

18. The method of claim 17, wherein the device is a stent, a shunt, a pacemaker lead, an implantable defibrillator, a suture, a staple, or a perivascular wrap, or a pliable sheet or membrane, which can substantially conform to the contours of a wound site comprising said tissue.

19. The method of claim 5, further comprising administering a pro-angiogenic factor, a statin, and/or a an inhibitor of an endogenous angiostatic factor to said subject.

20. A method of enhancing endothelial repair at site of vascular injury, comprising administering an effective amount of nitric oxide to said site.

21. An implantable medical device suitable for use in a human, wherein a coating or film comprising a nitric oxide releasing agent or nitric oxide generating agent is disposed on a surface of said device; wherein said device increases angiogenesis by at least 10%, as compared with angiogenesis generated by a device comprising a coating or film without said nitric oxide releasing agent or nitric oxide generating agent.

22. The device of claim 21 which is a stent, shunt, pacemaker lead, implantable defibrillator, suture, staple, or perivascular wrap.
